(19) United States
(12) Patent Application Publication
(10) Pub. No.: US 2005/0065086 A1
(43) Pub. Date: Mar. 24, 2005

(54) Use of peptide-drug conjugation to reduce inter-subject variability of drug serum levels

(75) Inventors: Randal Kirk, Radford, VA (US); Thomas Piccariello, Blacksburg, VA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: New River Pharmaceuticals Inc., Radford, VA (US)

(21) Appl. No.: 10/923,089

(22) Filed: Aug. 23, 2004

Related U.S. Application Data
(63) Continuation-in-part of application No. PCT/US03/05527, filed on Feb. 24, 2003.
(60) Provisional application No. 60/358,382, filed on Feb. 22, 2002.
(60) Provisional application No. 60/362,083, filed on Mar. 7, 2002.

(51) Int. Cl. A61K 38/17

(52) U.S. Cl. 514/12

(57) ABSTRACT

The present invention provides compositions and methods to decrease inter-patient variability particularly with respect to the systemic concentration of a drug. More particularly the invention relates to oral drugs which are conjugated to peptides or related carriers which alter release characteristics as compared to the analogous free drug.

### CROSS RELATED APPLICATIONS

This application claims benefit under 35 U.S.C. 120 and is a continuation-in-part application of PCT application No. PCT/US03/05527 filed Feb. 24, 2003, which claims priority under 35 U.S.C. 119(e) to U.S. Provisional Application 60/358,382 filed Feb. 22, 2002, and U.S. Provisional application 60/362,083 filed Mar. 7, 2002, all of which are hereby incorporated by reference in their entirety.

### FIELD OF THE INVENTION

The present invention is directed to the synthesis of amino acid polymers conjugated with drug molecules and the use of these conjugates to deliver drugs into the serum in a manner by which the variability between individuals is less than that seen when the drugs are given as monomers.

The extent of absorption for orally administered drugs is critical in determining the serum level or the concentration of the drug in the systemic circulation. Once in the bloodstream the drug molecule may experience a variety of fates including binding to serum proteins, distribution to its locus of action (the desired fate) as well as tissue reservoirs, biotransformation or metabolism and, ultimately, excretion. These fates are preceded by the initial process of absorption. Although the oral route is generally considered to be the safest and most convenient route, it does impart a relatively high degree of variability. One of the reasons that the oral route is safe is because drugs in the gastrointestinal (GI) tract may be metabolized by enzymes (from the intestinal flora, the mucosa and the liver) prior to their arrival into the general circulation. The metabolism of drugs occurring between absorption and systemic circulation is referred to as the “first pass effect.”

In some instances it is possible to measure serum levels after a set dose and calculate relevant parameters but this is not done routinely. The optimization of dosing regimens is more commonly determined by the more practical method of measuring a therapeutic drug effect and adjusting dosage until the desired effect is achieved. In cases where the therapeutic effect is more subjective, such as many of the drugs commonly used to treat psychiatric disorders, doses may be adjusted to avoid adverse effects such as nausea or dizziness. In some cases, it can be argued that drug dose optimization receives less attention than it deserves in day to day clinical practice. At any rate, since therapeutic drug monitoring is often difficult outside the hospital, any help in decreasing the variation between patients will be of practical significance in the determination of dosing instructions. This is especially true for new medications which are just being started for a particular patient.

### SUMMARY OF THE INVENTION

The invention comprises of a drug molecule covalently bonded to a biopolymer such as a peptide. After oral administration, digestive enzymes such as pancreatic proteases catalyze hydrolysis of the peptide leading to absorption of the drug. This absorption occurs in a manner so as to produce less variable serum drug levels between patients than that with the drug alone.

It is another embodiment of the present invention that the active agents may be combined with peptides of varying amino acid content to impart specific physicochemical properties to the conjugate including, molecular weight, size, functional groups, pH sensitivity, solubility, three dimensional structure and digestibility in order to provide desired performance characteristics. Similarly, a variety of active agents may also be used with specific preferred peptides to impart specific performance characteristics. Significant advantages with respect to the stability, release and/or adsorption characteristics of the active agent that are imparted through the use of one or more of the 20 naturally occurring amino acids are manifest in the peptide physicochemical properties that impart specific stability, digestibility and release properties to the conjugates formed with active agents.

In another embodiment of the invention is the concept that the amino acids that make up the carrier peptide are a tool set such that the carrier peptide can conform to the pharmacological demand and the chemical structure of the active agent such that maximum stability and optimal performance of the composition are achieved.

In another preferred embodiment the amino acid chain length can be varied to suit different delivery criteria. For delivery with increased bioavailability, the active agent may be attached to a single amino acid to eight amino acids, with the range of two to five amino acids being preferred. For modulated delivery or increased bioavailability of active agents, the preferred length of the oligopeptide is between two and 50 amino acids in length. For conformational protection, extended digestion time and sustained release, preferred amino acid lengths may be between 8 and 400 amino acids. In another embodiment, the conjugates of the present invention are also suited for both large and small molecule active agents. In another embodiment of the present invention, the carrier peptide controls the solubility of the active agent-peptide conjugate and is not dependant on the solubility of the active agent. Therefore, the mechanism of sustained or zero-order kinetics afforded by the conjugate-drug composition avoids irregularities of release and cumbersome formulations encountered with typical dissolution controlled sustained release methods.

In another preferred embodiment, the active agent conjugates can incorporate selected adjuvants such that the compositions interact with specific receptors so that targeted delivery may be achieved. These compositions provide targeted delivery in all regions of the gut and at specific sites along the intestinal wall. In another preferred embodiment, the active agent is released as the reference active agent from the peptide conjugate prior to entry into a target cell. In another preferred embodiment, the specific amino acid sequences used are not targeted to specific cell receptors or designed for recognition by a specific genetic sequence. In a more preferred embodiment, the peptide carrier is designed for recognition and/or is not recognized by tumor promoting cells. In another preferred embodiment, the active agent delivery system does not require that the active agent be released within a specific cell or intracellularly. In a preferred embodiment the carrier and/or the conjugate do result is specific recognition in the body. (e.g. by a cancer cell, by primers, for improving chemotactic activity, by sequence for a specific binding cite for serum proteins (e.g. kinins or eicosanoids).

In another embodiment the active agent may be attached to an adjuvant recognized and taken up by an active transporter. In a more preferred example the active transporter is not the bile acid active transporter. In another embodiment, the present invention does not require the attachment of the active agent to an adjuvant recognized and taken up by an active transporter for delivery. In a another embodiment the adjuvant provides an alternate mechanism of transport that overcomes the limitations of passive diffusion. Further the facilitation of active transport can be facilitated by the peptide carrier, the adjuvant or the combination.

In preferred embodiments the active agent conjugate is not bound to an immobilized carrier, rather it is designed for transport and transition through the digestive system.

It is a further embodiment of the invention that the reduce variability due to the increase stability of the drug conjugate by virtue of the protective effect the peptide has on the active agent. This protective effect can be imparted to those active agents that are acid labile and otherwise would degrade in the stomach. In addition the carrier peptide can protect the active agent from enzymes secreted by the stomach or the pancreas where the active agent is protected until it is absorbed and then release by peptidases within in the intestinal epithelial cells.

While microspheres/capsules may be used in combination with the compositions of the invention, the compositions are preferably not incorporated with microspheres/capsules and do not require further additives to improve sustained release or modulate adsorption.

In a preferred embodiment the active agent is not a hormone, glutamine, methotrexate, daunorubicin, a trypsin-kallikrein inhibitor, insulin, calmodulin, calcitonin, L-dopa, interleukins, gonadoliberin, norethindrone, tolmetin, valacyclovir, taxol, or silver sulfadiazine. In a preferred embodiment wherein the active agent is a peptidic active agent it is preferred that the active agent is unmodified (e.g. the amino acid structure is not substituted).

In a preferred embodiment the invention provides a carrier and active agent which are bound to each other but otherwise unmodified in structure. In a more preferred embodiment the carrier, whether a single amino acid, dipeptide, tripeptide, oligopeptide or polypeptide is comprised only of naturally occurring amino acids.

In a preferred embodiment the carrier is not a protein transporter (e.g. histone, insulin, transferrin, IGF, albumin or prolactin), Ala, Gly, Phe-Gly, or Phe-Phe. In a preferred embodiment the carrier is also not an amino acid copolymerized with a non-amino acid substitute such as PVP, a poly(alkylene oxide)amino acid copolymer, or an alkyloxycarbonyl(polyaspartate/polyglutamate) or an aryloxycarbonylmethyl (polyaspartate/polyglutamate).

In a preferred embodiment neither the carrier or the conjugate is used for assay purification, binding studies or enzyme analysis.

In another embodiment, the carrier peptide allows for multiple active agents to be attached. The conjugates provide the added benefit of allowing multiple attachments not only of active agents, but of active agents in combination with other active agents, or other modified molecules which can further modify delivery, enhance release, targeted delivery, and/or enhance adsorption. In a further embodiment, the conjugates may also be combined with adjuvants or be microencapsulated.

In a preferred embodiment the invention provides a carrier and active agent which are bound to each other but otherwise unmodified in structure. This embodiment may further be described as the carrier having a free carboxy and/or amine terminal and/or side chain groups other than the location of attachment for the active agent. In a more preferred embodiment the carrier, whether a single amino acid, dipeptide, tripeptide, oligopeptide or polypeptide comprises only naturally occurring amino acids.

### BRIEF DESCRIPTION OF THE DRAWINGS

FIG. 1 illustrates a typical release profile for reference drug v. a peptide conjugate drug of the present invention;

FIG. 2 illustrates a graph of factors which effect bioavailability taken from Amindon et al.;

FIG. 3 illustrates basolateral T4-conjugate concentrations as compared to T4 alone and control (Basolateral T4 concentrations);

FIG. 4 illustrates T4-conjugate concentration for both apical and basolateral concentrations;

FIG. 5 illustrates PolyT4 (T4-conjugate) vs. T4 sodium Mean Total T4 (TT4) Serum Concentrations and Delta (TT4);

FIG. 6 illustrates PolyT3 vs. T3 sodium Mean Total T3 (TT3) Serum Concentrations and Delta (TT3);

FIG. 7 illustrates Polythroid vs. T4 sodium plus T3 sodium vs. T3 sodium Total T3 Serum Concentration Curves;

FIG. 8 illustrates Chemical Structures of Phosphorylated AZT and Thymidine;

FIG. 9 illustrates AZT vs. LeuGlu/AZT Conjugate Serum Concentration Curves;

FIG. 10 illustrates a clinical trial of Poly T₃ vs. T₃ monomer in humans (variability in serum T₃ levels).

### DETAILED DESCRIPTION OF PREFERRED EMBODIMENTS

In quantifying drug absorption it is useful to apply the term bioavailability. This is defined as the fraction (F) of the dose that reaches the systemic circulation. Thus, in the extreme cases, F=0 in drugs which are not absorbed at all in the GI tract while for drugs that are completely absorbed (and not metabolized by a first pass effect) F=1. The bioavailability can be calculated from the area under the curve (AUC) of the serum level vs. time plot. It depends on many factors and some of these factors differ between normal individuals. The coefficient of variation (CV) is typically used to express the variability in bioavailability. This value is obtained by expressing the standard deviation as a percentage of the arithmetic mean.

For example, in a study of the antiseizure drug, gabapentin, Gidal and coworkers found the intersubject CV for the AUC was 22.5% after oral administration. Similarly, for the cholesterol lowering agent cerivastatin, the interindividual variability in AUC is between 30% and 40%. The CV for morphine was found in a study of cancer patients to be 50%. A high degree of variability related to the first pass effect may account for the high CV value morphine. In general, the CV for the bioavailability of many drugs is about 20%. This is not unusual in pharmacokinetics since other parameters may vary by an even greater amount. For example, the CV is about 30% for the steady-state volume of distribution (Vss) and 50% for the rate of clearance (CL). However, a modification in drug delivery that would minimize the variability of bioavailability would be therapeutically valuable. Ideally, the values for all of these pharmacokinetic parameters for individuals being prescribed medications are known by the physician but this is very rarely true.

In 1998 it was reported by Stavchansky and Pade that many of the drugs studied had a linear correlation between percent absorbed in humans and permeability except furosemide. Interestingly, chlorothiazide, which is closely related structurally to furosemide, had low permeability and low absorption in humans that correlated well with the other drugs. (See, Link between Drug Absorption Solubility and Permeability Measurements in Caco-2 Cells; J. of Pharm. Sci. Vol. 87, No. 12:160407 (1998)). Furosemide's absorption was higher than predicted by the plot, in fact its permeability was lower than chlorothiazide. It stands to reason that furosemide may be transported by a different mechanism than chlorothiazide even though they are very similar chemically. In addition, the study also showed that furosemide, chlorothiazide and cimetidine may have active efflux mechanisms opposing passive absorption. Thus a study on the improvement of absorption of chlorothiazide to overcome its poor permeability and solubility would serve as a significant advancement to its overall performance and may also reduce the absorption variability found with diuretics.

Variability can be defined as lower standard deviation or reduction in the number of outliers. This translates directly into a reduction of the number of adverse events that occur with the use of a given pharmaceutical. It is an embodiment of this invention that the reduction in inter-subject variability be accomplished by reducing the number of outliers for absorption.

The variation in biological response of individual patients to a given dose of a drug has multiple causes. A normal population of patients will respond to various degrees to a drug that is present at a specific concentration in the blood. The present invention does not pertain to that source of difference between patients. The focus here is the variability between patients in the resulting blood level after the oral administration of a given dose. Specifically, it is the absorption of the drug from the gastrointestinal tract. Critical to this process are the concepts of diffusion and transport. The movement of a drug from one place to another within the body is referred to as transport. This process typically involves the movement across a biological membrane and may occur by any one or the combination of the following types of diffusion.

Simple Nonionic Diffusion and Passive Transport—This type of movement is used to describe the random motion of uncharged molecules through a field devoid of an electrical gradient. The change in the net quantity of drug transported across the membrane (O) over time is given by Fick's Law of Diffusion: dQ/dT=DA(C₁-C₂)/x; where:

D=diffusion coefficient, A=area; C₁ and C₂ are concentrations on either side of a membrane and x is the thickness of the membrane. The membrane factors are typically combined into one constant called P, the permeability constant or coefficient. Thus, passive diffusion can be described by the following equation, dQ/dt=P(C₁-C₂). The movement of the drug across the concentration gradient continues in a first order process until the concentrations across the membrane are equal.

Ionic or Electrochemical Diffusion—Ionized drug molecules will be distributed according to an electrochemical gradient in addition to moving from a higher to a lower concentration. Thus, negatively charged drugs will diffuse differently than positively charged drugs.

Facilitated Diffusion—This describes movement across a biological membrane which is accelerated relative to simple diffusion. A special carrier molecule within the membrane is thought to combine with the drug on one side and move it, along its electrochemical gradient, to the other side. There, the drug dissociates from the carrier which is then free to repeat the process.

Active Transport—In contrast to facilitated diffusion, this process involves an energy-dependent movement of a drug through a biological membrane against an electrochemical gradient. The transport system typically shows a requirement for a specific chemical structure of the transported molecule and competes for molecules that are closely related with respect to key elements of the chemical structure. There are seven known intestinal transport systems classified according to the physical properties of the transported substrate. They include the amino acid, oligopeptide, glucose, monocarboxylic acid, phosphate, bile acid, and the P-glycoprotein transport systems and each has its own associated mechanism of transport. The mechanisms can depend on hydrogen ions, sodium ions, binding sites, or other cofactors.

Pinocytosis and Exocytosis—These processes describe the movement of substances into and out of a cell, respectively, through a type of phagocytosis. The cell membrane invaginates so as to contain the drug inside a pinched off vesicle and transports the drug across the membrane. This type of transport is thought to be important in the gut where it may be involved in the absorption of macromolecules and larger particles such as certain proteins.

Improved Absorption—Physicochemical and biological factors that influence the extent of drug absorption from the gastrointestinal (GI) tract include solvation, hydrogen bonding, conformational changes, pH, pKa, log P, metabolism and extrinsic and intrinsic factors. Inherent in each drug are combinations of these factors that dictate specific mechanisms of absorption. For the most part drugs are absorbed by passive transport, ionic diffusion, facilitated diffusion, active transport or pinocytosis. In addition, where drugs have a low degree of permeability, highly variable bioavailability is frequently observed. Either improving the permeability or promoting an active transport mechanism should enhance the bioavailability of this class of drug. For those drugs that rely primarily on active transport (e.g. DOPA, levothyroxine, liothyronine) improving the drug's solubility or providing the drug with an alternate transport pathway should enhance absorption, as well.

Lower Peak Values—One of the fundamental considerations in drug therapy involves the relationship between blood levels and therapeutic activity. For most drugs, it is of primary importance that serum levels remain between a minimally effective concentration and a potentially toxic level. In pharmacokinetic terms, the peaks and troughs of a drug's blood levels ideally fit well within the therapeutic window of serum concentrations.

Low Peak Values for certain therapeutic agents, this window is so narrow that dosage formulation becomes critical. Such is the case with the drug, digoxin, which is used to treat heart failure. Therapeutic blood levels include the range between 0.8 ng/mL (below which the desired effects may not be observed) and about 2 ng/mL (above which toxicity may occur). Among adults in whom clinical toxicity has been observed, two thirds have serum digoxin concentrations greater than 2 ng/mL. Furthermore, adverse reactions may increase dramatically with small increases above this maximum level. For example, digoxin-induced arrhythmias occur at 10%, 50%, and 90% incidences at serum drug levels of 1.7, 2.5 and 3.3 ng/mL, respectively.

After the oral administration of digoxin, an effect will usually be evident in 1-2 hours with peak effects being observed between 4 and 6 hours. After a sufficient time, the concentration in plasma and the total body store is dependent on the single daily maintenance dose. It is critical that this dose be individualized for each patient. Having a dosage form of digoxin that provides a more consistent serum level between doses is therefore useful.

Another example is provided by the β-blocker atenolol. The duration of effects for this commonly used drug is usually assumed to be 24 hours. However, at the normal dose range of 25-100 mg given once a day, the effect may wear off hours before the next dose begins acting. For patients being treated for angina, hypertension, or for the prevention of a heart attack, this may be particularly risky. One alternative is to give a larger dose than is necessary in order to get the desired level of action when the serum levels are the lowest. This risks side effects related to excessive concentrations in the initial hours of the dosing interval. At these higher levels, atenolol loses its potential advantages β-1 selectivity and adverse reactions related to the blockade of β-2 receptors become more significant. That could be avoided with more constant atenolol levels following PolyAtenolol administration.

Reduced Variability—There have been several models proposed to predict the bioavailability of drugs through the gastrointestinal tract. The model proposed by Amidon, et. al. provides a convenient way to generate visual algorithms. (See, Amidon, GL, Lennernas, H; Shah, VP, Crison, JR (1995). “A Theoretical Basis for a Biopharmaceutic Drug Classification: The Correlation of in Vitro and in Vivo Bioavailability.” Pharm. Res., 12 (3), 413-20; Amidon, GL, Oh, D-M, Curl, RL (1993). “Estimating the Fraction Dose Absorbed from Suspensions of Poorly Soluble Compounds in Humans: A Mathematical Model.” Pharm. Res., 10 (2), 264-70.). The Amidon model uses three key dimensionless variables to predict drug absorption or the fraction of drug absorbed (F). The first variable, absorption number (An), is proportional to the effective permeability (P_{eff}) of the drug and the volumetric flow rate of the intestine (tᵣₑₛ/R) and is determined by the equation: An=(P_{eff}·tᵣₑₛ)/R. The second variable, dose number (Do), is a function of the dose (M₀), the drug solubility (Cₛ) and volume of water taken with the drug (V₀) and is determined by the equation: Do=M₀/(Cₛ·V₀). The third variable, dissolution number (Dn), includes diffusivity (D), solubility (Cₛ), intestinal transit time (tᵣₑₛ), particle size (r) and density (ρ) and is determined by the equation: Dn=(3D·Cₛ·tᵣₑₛ)/(r²·ρ).

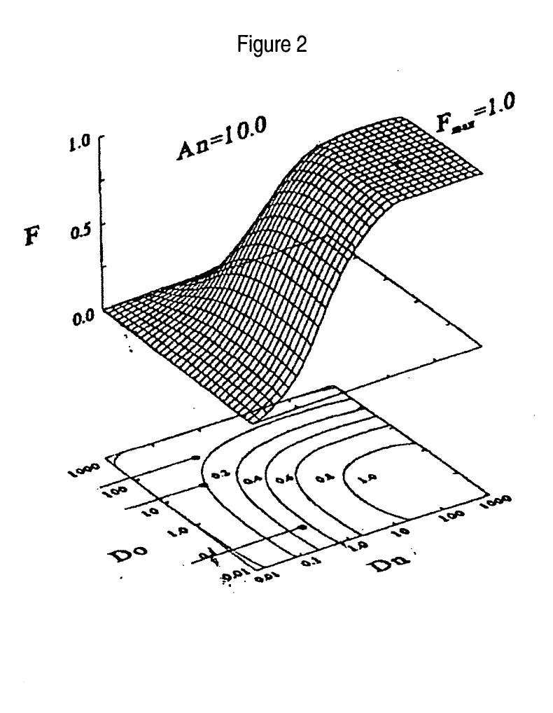

The F can be estimated by solving these and other equations simultaneously, the description of which will not to be discussed here. Suffice it to say that a contour plot of estimated F versus Dn and Do with a given An can be generated. FIG. 2 shows a typical profile of a highly permeable drug with An=10. (FIG. 2 is from Pharm. Res., 12(3), 416). As can be seen the slope of the curve is greatest in the critical regions of Do (10-100) and Dn (0.2-2). This critical region corresponds to an extent of absorption of the drug that is most variable. For An values lower than 10 the slopes in the critical region are steeper and the area for Fₘₐₓ is less. Thus, the bioavailability of a drug could be enhanced by increasing its An, which can be accomplished by promoting an active transport mechanism.

To illustrate this point, table 1 shows the different values of An, Do and Dn that were derived to get 90% absorption or F=90%. The tabulated data shows that increasing An reduces the change in Dn across a range of Do values. For example, at An=2.0, the change in Dn is 2.06−1.87=0.19 with Do ranging from 0.1 to 0.5. Comparatively, at An=7.0, the change in Dn is 1.32−1.28=0.04 over the same range of Do. This means that a drug with a given Dn value, its Fₘₐₓ can be retained at a wider range of Do values as the An number is increased. In other words, the higher the An value of a drug the more flexible is the dosing of the drug and the lower the variability in the fraction absorbed.

TABLE 1
Values of Absorption number (An), Dose number (Dn) and
Dissolution number (Dn) for a Fraction dose absorbed of 90%.

| An | Do | Dn |
|---|---|---|
| 1.15 | —^{a} | —^{b} |
| 2.0 | 0.1 | 1.87 |
| 2.0 | 0.5 | 2.06 |
| 2.0 | 1.0 | 2.38 |
| 2.0 | 4.4 | —^{b} |
| 3.0 | 0.1 | 1.49 |
| 3.0 | 0.5 | 1.59 |
| 3.0 | 1.0 | 1.73 |
| 3.0 | 5.0 | 6.29 |
| 3.0 | 6.7 | —^{b} |
| 5.0 | 0.1 | 1.33 |
| 5.0 | 0.5 | 1.39 |
| 5.0 | 1.0 | 1.46 |
| 5.0 | 5.0 | 2.44 |
| 5.0 | 10.0 | 13.94 |
| 5.0 | 11.1 | —^{b} |
| 7.0 | 0.1 | 1.28 |
| 7.0 | 0.5 | 1.32 |
| 7.0 | 1.0 | 1.36 |
| 7.0 | 5.0 | 1.89 |
| 7.0 | 10.0 | 3.64 |
| 7.0 | 15.6 | —^{b} |

^{a}No Do limit is assumed
^{b}No Dn limit is assumed

The thyroid hormone T4 can serve as an example of how increasing the Dn of a drug can reduce the variability of drug absorbance. (For those drugs with critical Do values, decreasing the Do would, likewise, reduce the variability). Estimating T4's Cs to be 6.9 μg/ml, assuming V₀ to be 250 ml and using a typical dose of 100 μg the Do of T4 can be estimated to be 0.057. Since orally administered thyroid hormones are, most likely, actively transported across the intestinal epithelia it can be assumed that the An of T4 is approximately 10. This is the experimentally determined An for glucose, which is known to be actively transported. From the contour plot in FIG. 2 and the reported bioavailability of T4, the Dn of T4 can be estimated to be between 0.2 and 2. For Dn=1, Cₛ=6.9 μg/ml, tᵣₑₛ=240 min., r=25 μm and ρ=1000 mg/mil, D of T4 is estimated to be 1.21×10⁻³ cm²/min., which is a relatively high number and thus a Dn number of greater than 1 for T4 is unlikely unless Cₛ is increased. Keeping all other variables equal, increasing the Cₛ of T4 to 69 μg/ml would increase the Dn to 10 and decrease the Do to 0.0057. This puts the F for T4 near the upper plateau of the contour plot (i.e. Fₘₐₓ) where the absorption is maximal and its variability is minimal.

Assume that the An of T4 was equal to 7. Then in order for T4 to be 90% absorbed its Dn would need to be approximately 1.3 which would be difficult to achieve. So if T4's An=7, Dn=1 and Do=0.057 then the F of T4 would be well below the 48% reported. In any event, increasing the bioavailability of a drug, either by increasing Dn or An or by decreasing Do, reduces the variability of its absorbance.

With these types of transport in mind and the above criteria, it is clear why each of the following factors can influence absorption of drugs: concentration, physical state of formulation, dissolution rate, area of absorbing surface, vascularity and blood flow, gastric motility and emptying as well as solubility. One way of enhancing absorption into cells is to attach drugs to peptides. In terms of the previous discussion, peptide drug conjugates may serve to engage facilitated and active transport processes and pinocytosis which would not otherwise be observed in drug absorption.

There is evidence that certain compounds are absorbed through the intestinal epithelia efficiently via specialized transporters. There are seven known intestinal transport systems classified according to the physical properties of the transported substrate. They include the amino acid, oligopeptide, glucose, monocarboxic acid, phosphate, bile acid and the P-glycoprotein transport systems and each has its own associated mechanism of transport. The mechanisms can depend on hydrogen ions, sodium ions, binding sites or other cofactors. The invention also allows targeting the mechanisms for intestinal epithelial transport systems to facilitate absorption of active agents.

The entire membrane transport system is intrinsically asymmetric and responds asymmetrically to chiral compounds such as amino acids. Thus, one can expect that excitation of the membrane transport system will involve some sort of specialized adjuvant resulting in the enhanced transport of active agents across biological membranes. Suitable adjuvants, for example, include: papain, which is a potent enzyme for releasing the catalytic domain of aminopeptidase-N into the lumen; glycorecognizers, which activate enzymes in the brush border membrane; and bile acids, which have been attached to peptides to enhance absorption of the peptides.

Caco-2 or other intestinal epithelial model systems (such as HT29-H goblet cells in culture) may be used to predict intestinal drug absorption. Early studies using these model systems demonstrate that drugs absorbed via the passive transcellular absorptive pathway are easily studied in these model systems due to the their requirement for relatively less absorptive surface (found in culture models as compared to the extensively folded intestinal lining) area. In addition, the Caco-2 cell model has been optimized for the re-differentiation of the tumor cells and therefore re-expression of key epithelial markers (this is accomplished by plating the cells on collagen fibril scaffold and supplementing the cells in a defined cytokine cocktail). The HT29 cells, however, can produce mucus but fail to express other differentiation markers for epithelial cells and are generally regarded as a less reliable model for bioabsorption.

Drugs that are absorbed through a passive paracellular route (usually molecular size limited) are not efficiently absorbed in the Caco-2 model, likely due to this models relatively fewer pores in their tight junctions. However, the correlation between the in vitro absorption of these molecules is qualitatively the same as the absorption in vivo.

Drugs that are absorbed using an active transport process appear to require characterization of the transport process to fully understand any in vitro/in vivo correlations. For example, Caco-2 cells do not transport L-dopa very well unlike its in vivo rapid and efficient absorption via a carrier for large neutral amino acids. This is attributed to the low expression of this carrier in culture. Other compounds, which utilize active transport mechanisms, appear to correlate better with in vivo absorption, suggesting that the transport mechanism should be defined before the correlation.

Therefore preferably the active agent conjugates are absorbed via paracellular or active transport mechanisms. The Caco-2 model has been optimized for the re-expression of cell associated proteases so the potential for release of the pro-drug (conjugate) is greater. the conjugates may also facilitate binding to the cell surface via cell surface receptors such as di- and tri-peptide transporters or some unknown, but specific, receptor which provides a mechanism for consistency of dosing. Further, the re-differentiated Caco-2 cells are capable of re-expressing the correct repertoire of cell surface molecules. Below are three potential mechanisms for release/absorption to produce reproducible uptake:
(1) Facilitated binding to the cell surface via the pro-drug moiety and the release by the cell surface associated proteases.
(2) Facilitated binding to the cell surface via the pro-drug moiety and endocytosis followed by release in the lysosomal environment of the endocytotic vesicles.
(3) Active transport of small dimer/trimer based pro-drugs and release either in lysosomal compartments or by serum proteases.

One embodiment of the invention provides methods for determining how the conjugation of a drug to a single amino acid, dipeptide, tripeptide, oligopeptide and/or a peptide alters absorption. In a preferred embodiment the active agent is furosemide which was synthesized by conjugating furosemide to each of the twenty common amino acids used in protein synthesis. In a preferred embodiment the Furosemide Dipeptide Serine Conjugates are selected from Ile-Ser(Furosemide)-Ome; Glu-Ser(Furosemide)-Ome and Phe-Ser(Furosemide)-OH. The addition of each amino acid conjugate may then be tested for any affects on the absorption of furosemide through the Caco-2 cells. When facilitated transport is observed, additional experiments may then be conducted to evaluate the process through which facilitation occurs. To further alter the effect of the amino acid conjugate additional amino acids may be conjugated to alter the pharmacokinetic parameters.

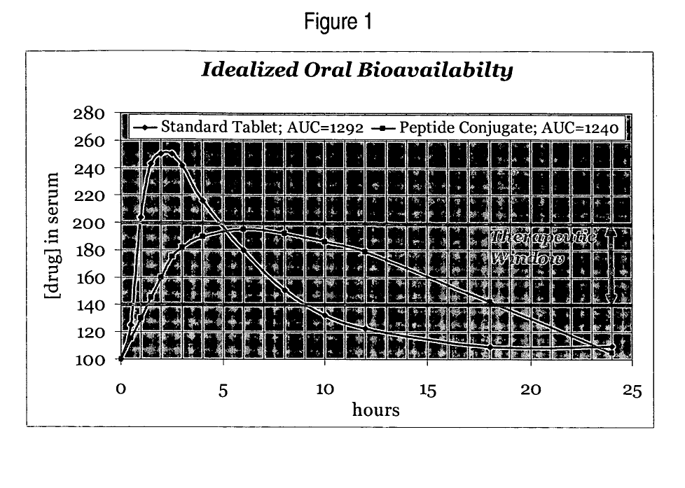

The invention also provides a method for controlling release of an active agent from a composition wherein the composition comprises a peptide, the method comprising covalently attaching the active agent susceptible to peptide controlled release to the peptide. It is a further embodiment of the invention that enhancement of the performance of active agents from a variety of chemical and therapeutic classes is accomplished by extending periods of sustained blood levels within the therapeutic window. For a drug where the standard formulation produces good bioavailability, the serum levels may peak too fast and too quickly for optimal clinical effect as illustrated in FIG. 1. Designing and synthesizing a specific peptide conjugate that releases the active agent upon digestion by intestinal enzymes mediates the release and absorption profile thus maintaining a comparable area under the curve while smoothing out active agent absorption over time.

Conjugate prodrugs of the invention afford sustained or extended release to the parent compound. Sustained release typically refers to shifting absorption toward slow first-order kinetics. Extended release typically refers to providing zero-order kinetics to the absorption of the compound. Bioavailability may also be affected by factors other than the absorption rate, such as first pass metabolism by the enterocytes and liver, and clearance rate by the kidneys. Mechanisms involving these factors require that the drug-conjugate is intact following absorption. The mechanism for timed release may be due to any or all of a number of factors. These factors include: 1) gradual enzymatic release of the parent drug by luminal digestive enzymes, 2) gradual release by surface associated enzymes of the intestinal mucosa, 3) gradual release by intacellular enzymes of the intestinal mucosal cells, 4) gradual release by serum enzymes, 5) conversion of a passive mechanism of absorption to an active mechanism of uptake, making drug absorption dependent on the Km for receptor binding as well as receptor density, 6) decreasing the solubility of the parent drug resulting in more gradual dissolution 7) an increase in solubility resulting in a larger amount of drug dissolved and therefore absorption over a longer period of time due to the increased amount available.

The potential advantages of enzyme mediated release technology extend beyond the examples described above. For those active agents that can benefit from increased absorption, it is an embodiment of this invention that this effect is achieved by covalently bonding those active agents to one or more amino acids of the peptide and administering the drug to the patient as stated earlier. The invention also allows targeting to intestinal epithelial transport systems to facilitate absorption of active agents. Better bioavailability, in turn, may contribute to lower doses being needed. Thus it is a further embodiment of the invention that by modulating the release and improving the bioavailability of an active agent in the manner described herein, reduced toxicity of the active agent can be achieved.

It is another embodiment of this invention that attachment of an amino acid, oligopepetide, or polypeptide may enhance absorption/bioavailability of the parent drug by any number of mechanisms, including conversion of the parent drug to a polymer-drug conjugate such that the amino acid-prodrugs may be taken up by amino acid receptors and/or di- and tri-peptide receptors (PEPT transporters). This may also hold true for polymer drug conjugates since by products of enzymatic activity in the intestine may generate prodrugs with 1-3 amino acids attached. Moreover, it is possible that other receptors may be active in binding and uptake of the prodrugs. Adding an additional mechanism(s) for drug absorption may improve its bioavailability, particularly if the additional mechanism is more efficient than the mechanism for absorption of the parent drug. Many drugs are absorbed by passive diffusion. Therefore, attaching an amino acid to the compound may convert the mechanism of absorption from passive to active or in some cases a combination of active and passive uptake, since the prodrug may be gradually converted to the parent drug by enzymatic activity in the gut lumen.

It is another embodiment of the invention that active agent efficiency is enhanced by lower active agent serum concentrations. It is yet another embodiment of the invention that conjugating a variety of active agents to a carrier peptide and, thereby sustaining the release and absorption of the active agent, would help achieve true once a day pharmacokinetics. In another embodiment of the invention, peaks and troughs can be ameliorated such as what could be achieved with more constant atenolol levels, for example, following administration of a peptide-atenolol conjugate.

In another embodiment of the present invention the amino acids used can make the conjugate more or less labile at certain pHs or temperatures depending on the delivery required. Further, in another embodiment, the selection of the amino acids will depend on the physical properties desired. For instance, if increase in bulk or lipophilicity is desired, then the carrier polypeptide will include glycine, alanine, valine, leucine, isoleucine, phenylalanine and tyrosine. Polar amino acids, on the other hand, can be selected to increase the hydrophilicity of the peptide. In another embodiment, the amino acids with reactive side chains (e.g., glutamine, asparagines, glutamic acid, lysine, aspartic acid, serine, threonine and cysteine) can be incorporated for attachment points with multiple active agents or adjuvants to the same carrier peptide. This embodiment is particularly useful to provide a synergistic effect between two or more active agents.

In another embodiment, the peptides are hydrolyzed by any one of several aminopeptidases found in the intestinal lumen or associated with the brush-border membrane and so active agent release and subsequent absorption can occur in the jejunum or the ileum. In another embodiment, the molecular weight of the carrier molecule can be controlled to provide reliable, reproducible and/or increased active agent loading.

Modulation is meant to include at least the affecting of change, or otherwise changing total absorption, rate of adsorption and/or target delivery as compared to the reference drug alone. Sustained release is at least meant to include an increase in the amount of reference drug in the blood stream for a period up to 36 hours following delivery of the carrier peptide active agent composition as compared to the reference drug delivered alone. Sustained release may further be defined as release of the active agent into systemic blood circulation over a prolonged period of time relative to the release of the active agent in conventional formulations through similar delivery routes.

The active agent is released from the composition by a pH-dependent unfolding of the carrier peptide or it is released from the composition by enzyme-catalysis. In a preferred embodiment, the active agent is released from the composition by a combination of a pH-dependent unfolding of the carrier peptide and enzyme-catalysis in a time-dependent manner. The active agent is released from the composition in a sustained release manner. In another preferred embodiment, the sustained release of the active agent from the composition has zero order, or nearly zero order, pharmacokinetics.

The present invention provides several benefits for active agent delivery. First, the invention can stabilize the active agent and prevent digestion in the stomach. In addition, the pharmacologic effect can be prolonged by delayed or sustained release of the active agent. The sustained release can occur by virtue of the active agent being covalently attached to the peptide and/or through the additional covalent attachment of an adjuvant that bioadheres to the intestinal mucosa. Furthermore, active agents can be combined to produce synergistic effects. Also, absorption of the active agent in the intestinal tract can be enhanced either by virtue of being covalently attached to a peptide or through the synergistic effect of an added adjuvant. The invention also allows targeted delivery of active agents to specific sites of action.

Throughout this application the use of “peptide” is meant to include a single amino acid, a dipeptide, a tripeptide, an oligopeptide, a polypeptide, or the carrier peptide. Oligopeptide is meant to include from 2 amino acids to 70 amino acids. Further, at times the invention is described as being an active agent attached to an amino acid, a dipeptide, a tripeptide, an oligopeptide, or polypeptide to illustrate specific embodiments for the active agent conjugate. Preferred lengths of the conjugates and other preferred embodiments are described herein. In another embodiment the number of amino acids is selected from 1, 2, 3, 4, 5, 6, or 7 amino acids. In another embodiment of the invention the molecular weight of the carrier portion of the conjugate is below about 2,500, more preferably below about 1,000 and most preferably below about 500.

Other embodiments of the invention are further illustrated by the examples and illustration which are not meant to limit the scope of the present invention.

### EXAMPLES

### Example 1

### Polythroid Enhances Absorption of T4 Across Caco-2 Monolayers

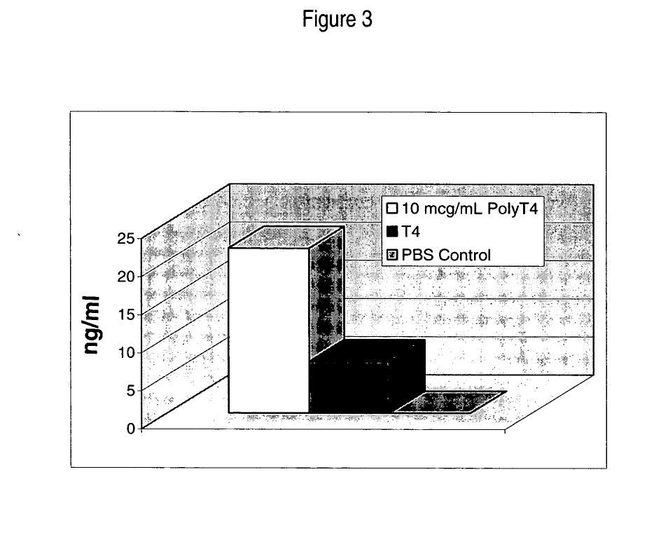

Absorption of T4 was monitored in the Caco-2 transwell system (n=4). Polythroid (10 micrograms) was added to the apical side of the transwells. T4 was added to the apical side at a concentration equal to the T4 content of Polythroid. A commercially available ELISA assay was used to determine the level of T4 in the basolateral chamber following incubation for 4 hours at 37° C. (FIG. 3). A significantly higher amount of T4 was absorbed from Polythroid as compared to Caco-2 cells incubated with the amount of T4 equivalent to that contained in the polymer.

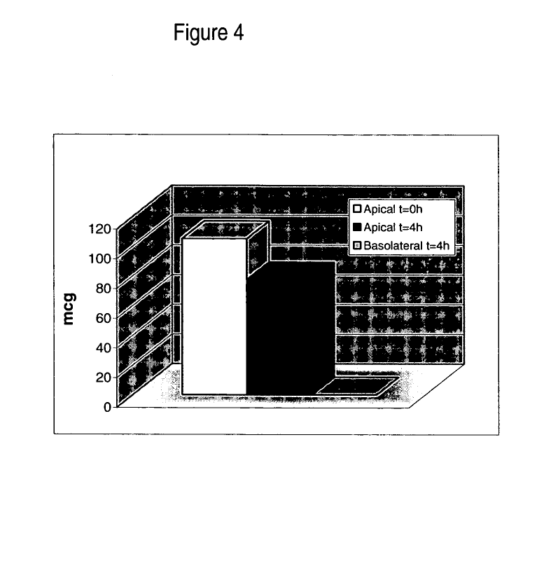

In order to determine if Polythroid itself crosses the Caco-2 monolayer we used the Polythroid specific ELISA to measure the amount of polymer in the basolateral chamber after incubation with Polythroid at a high concentration (100 micrograms). After 4 hours incubation, samples (n=4) from the basolateral side showed no reactivity in the ELISA (FIG. 4). The limit of detection for Polythroid is 10 ng, therefore, less than 1/10,000 of the Polythroid was absorbed. In conclusion, within the limits of ELISA detection, Polythroid does not cross the Caco-2 monolayer.

Our studies demonstrated the potential for reduced variability in patients through an in vitro experiment. Three ways to model reduced variability in patients through this type of in vitro experiment provide three options: (1) varying conditions in the Caco-2 transmembrane wells, (2) varying the cell line of the Caco-2, and/or (3) varying the peptide attached to the active agent. Given the fragility of the Caco-2 cells, option number one does not provide for a plausible demonstration due to the limits in experimental conditions available for testing. Option two, would make it difficult to demonstrate patient to patient variability because selecting for a new cell line would probably not express all the cellular transport mechanism required for absorption. As a result, only option three, the variation of the peptide carrier provides the necessary requirements. It would then be possible to test for the effectiveness and variability of different transporters and mechanisms of transport that are expressed in Caco-2 cells. Option three also identifies peptide transporters that are expressed in Caco-2 cells and by attaching active agents to the identified peptide one can demonstrate subject variability can be reduced by absorption across Caco-2 cells, provided the Caco-2 cells showed a statistically sound variability.

### Example 2

### PolyT4™ (Levothyroxine) and PolyT3™ (Liothyronine)

In the euthyroid state, the thyroid gland is the source of two iodothyronine hormones, tetraiodothyronine (T4) and triiodothyronine (T3). Both T4 and T3 play a key role in brain development, and in the growth and development of other organ systems. The iodo-hormones also stimulate the heart, liver, kidney, and skeletal muscle to consume more oxygen, directly and indirectly influence cardiac function, promote the metabolism of cholesterol to bile acids, and enhance the lipolytic response to fat cells. Hypothyroidism is the most common disorder of the thyroid and is manifested through the thyroid gland's inability to produce sufficient thyroid hormone.

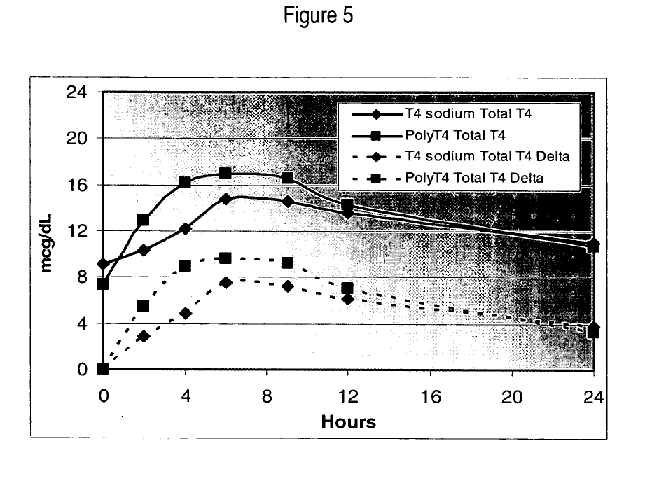

Currently, the most common treatment for hypothyroidism is the administration of levothyroxine sodium (or T4, sodium). There are several T4, sodium containing products on the market today, including Levothroid® (Forest), Unithroid® (Watson), Levoxyl® (Jones) and Synthroid® (Abbott). Studies have indicated that the bioavailability of T4 from T4 sodium varies between 48% and 80% thus making proper dosing difficult and often times requiring extensive titration periods. Increasing the absorption of orally administered T4 sodium should not only reduce the potential for overdosing but shorten the titration time for patients, as well. Thyroxine is an amino acid and, as such, can be attached to the C-terminus, N-terminus or both (interspersed) of the carrier peptide. Conceptually, by covalently attaching specific amino acids to T4, absorption of T4 is improved as demonstrated in rat feed and bleed studies where equipotent doses of T4, sodium and PolyT4 were compared. Eight separate studies were averaged and a plot of rat sera concentration of T4 vs. time revealed similar pharmacokinetics between the two compounds (FIG. 5). However, the Cₘₐₓ for the PolyT4 was greater than for the T4, sodium. Furthermore, analysis of the relative AUC's from the two compounds shows that PolyT4 was absorbed 37% better than T4, sodium (Table 2).

TABLE 2
T4 Performance Indices (PI)

| | | Percent T4 sodium* | | |
|---|---|---|---|---|
| Conjugate | No. of Studies* | AUC | Cmax | Deltamax |
| PolyT4 | 8 | 137 | 122 | 141 |

*The percentages depicted are average values.

The enhanced absorption may be explained by the use of an additional transport mechanism, such as one of the peptide transporters. Alternatively, the enhanced absorption may be due to the increased solubility of PolyT4 (70.5 μg/ml at pH 7.4) over T4, sodium (6.9 μg/ml at pH 7.4).

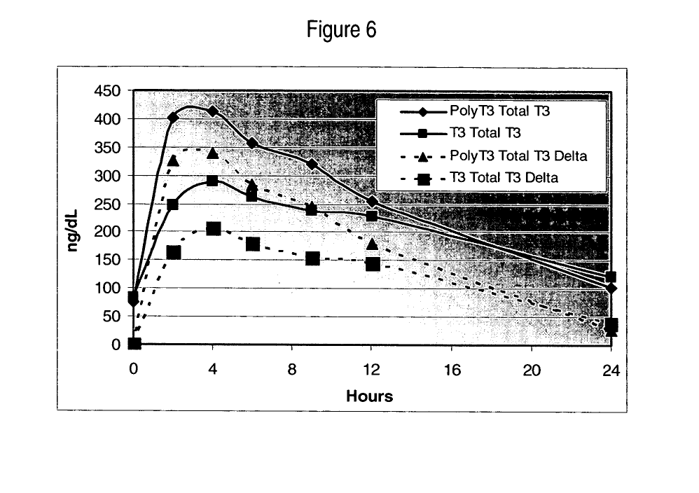

PolyT3 was subjected to the same series of rat feed and bleed studies as that of PolyT4 with similar results. FIG. 6 shows the relative pharmacokinetics between PolyT3 and T3, sodium in the rat model. As seen in Table 3, T3 is absorbed 150% from PolyT3 relative to T3, sodium.

TABLE 3
T₃ Performance Indices (P1)

| | | Percent of T₃ Sodium* | | |
|---|---|---|---|---|
| Conjugate | No. of Studies* | AUC | Cmax | Deltamax |
| PolyT₃ | 5 | 160 | 148 | 162 |

*The percentages depicted are average values.

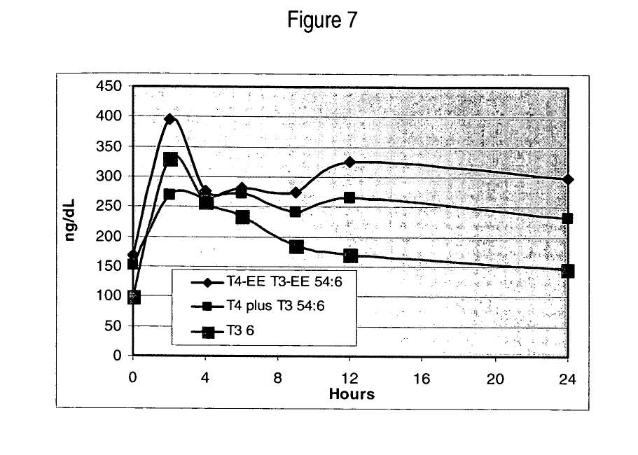

A T4/T3 combination product was designed to mimic the natural thyroid function in a euthyroid individual. A standard rat feed and bleed study demonstrated that the Cₘₐₓ of T3 was slightly lower from Polythroid than from T4/T3, sodium even though the AUC was greater. Further, by adjusting the Polythroid T3 dose to ⅔ of the T3 dose in the reference mixture, a dramatic decrease in Cₘₐₓ with concomitant equal AUC's was observed. (FIG. 7). Both DOPA and Carbidopa are amino acids and that possess similar chemical properties to T4 and T3. A DOPA-glutamic acid copolymer and a Carbidopa-glutamic acid copolymer were synthesized.

The T3 and T4 conjugates discussed in Examples 1 and 2 demonstrate:
(i) Enhanced absorption of both T3 and T4 which would reducing variability;
(ii) Reduced the Cₘₐₓ of T3 decreasing the likelihood of T3 spiking;
(iii) Delayed the release of T3 resulting in a longer duration of T3 serum levels.

### Example 3

### Poly AZT

PolyAZT was synthesized by the addition of AZT to a peptide containing a glutamic acid residue that was activated by bromotripyrrolidinophosphonium hexafluorophosphate (PyBrop). The attachment of other Other alcohol drugs may be attached using a similar procedure. For instance, other drugs attached through this procedure include, but are not limited to Quetiapine, Tolteridine, Acetaminophen and Tramadol.

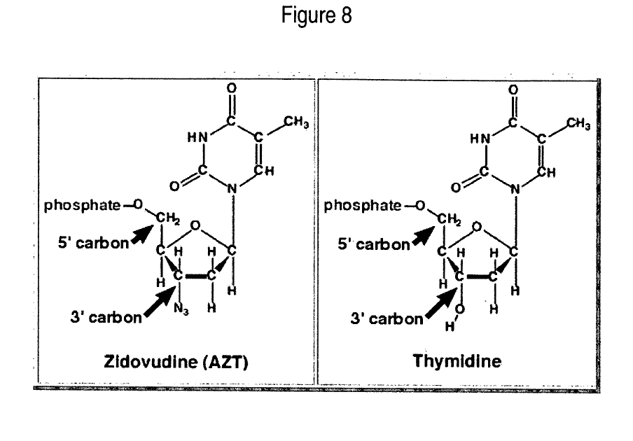

The peptide conjugate of AZT may have distinct clinical advantages over the parent drug. For example, an enhancement of intestinal absorption is known to occur for nucleoside analogs that are administered as amino acid ester prodrugs with increased the intestinal permeability of the parent nucleoside analog 3- to 10-fold (See, Han H, de Vrueh R L, Rhie J K, Covitz K M, Smith P L, Lee C P, Oh D M, Sadee W., Amidon G L (1998). “5′-Amino acid esters of antiviral nucleosides, acyclovir, and AZT are absorbed by the intestinal PEPT1 peptide transporter.” Pharm Res 15(8): 1154-9.). Another potential advantage is related to the activation of the drug once inside the cell. Similar to their nucleoside parents, analogs like AZT depend on intracellular phosphorylation at the 5′-OH group. (FIG. 8). Before they can inhibit reverse transcriptase, nucleoside analogs must undergo sequential phosphorylations catalyzed by specific kinases. The rate at which phosphorylation occurs depends on the concentration of substrate, in this case, AZT. The conjugate of AZT allows the change in concentration of the drug within the target cells over time in part because conjugate must be digested before it is absorbed. The amount of drug delivered to the cells is spread out over a longer time period. Therefore, the peptide conjugate is able to deliver the drug to cells at a concentration that more closely approximates levels needed by kinases to optimally phosphorylate the nucleoside and result in improved efficacy of a given dose over the dosing time interval.

Other nucleoside analogs can also be given as slowly digested peptide conjugates that retain lower peak serum concentrations (thus avoiding saturation of the kinases) and longer lasting moderate concentrations (closer to the levels that optimize rates of phosphorylation). This is especially valuable in nucleoside reverse transcriptase inhibitors since the same enzymes may catalyze the phosphorylation of different nucleoside analogs. When two or three nucleoside analogs are given simultaneously, as they often are in the “cocktails” currently administered, the maintenance of optimal substrate levels becomes even more important. Therefore the conjugates of the invention also allow for the administration of multiple nucleoside analogs as peptide conjugates to improve treatment efficacy.

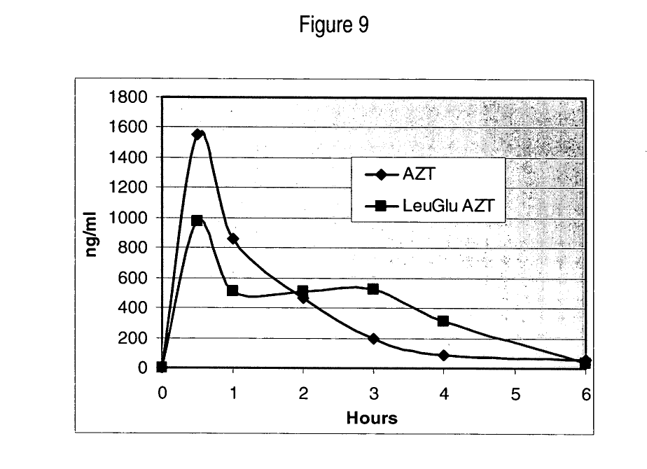

A peptide conjugate of AZT has the pharmacokinetic profile in rats (FIG. 9) that demonstrates plasma levels of AZT which remain elevated over twice as long as the parent drug given at equimolar doses while at the same time reducing the Cₘₐₓ by more than 35%. The PK of PolyAZT should therefore increase the phosphorylation efficiency of the drug and reduce side effects.

### Example 4

### Poly-T₃ (a Thyroid Hormone)

Liothyronine (T₃) is a naturally occurring hormone from the thyroid gland that is administered as a drug for the treatment of various endocrine disorders.

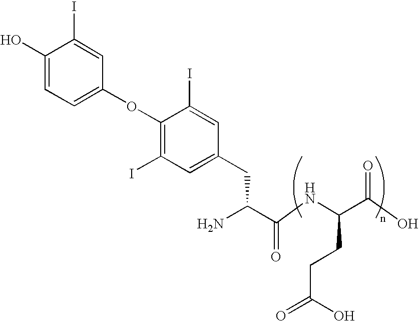

The synthetic polymer, poly-T₃, consists of poly-L-glutamic acid conjugated to a T₃ molecule. It is made by standard peptide chemistry and it is assayed for T₃ potency by total % I content. The chemical structure of one possible type of PolyT3 molecule is shown above.

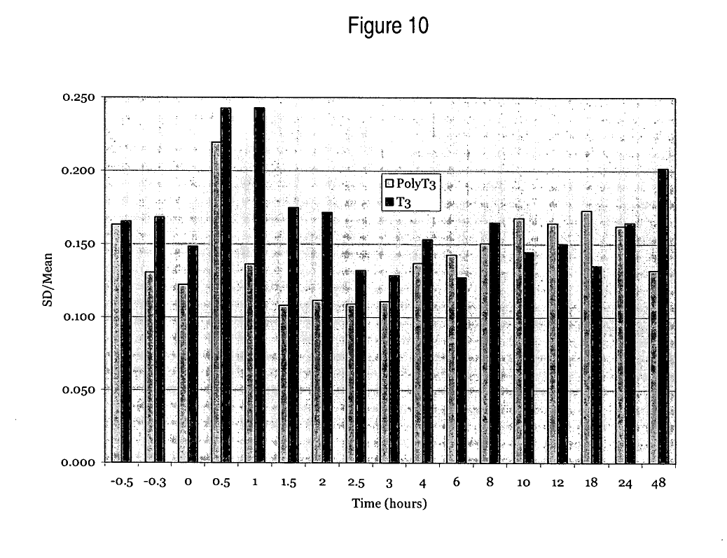

The data from a clinical trial of Poly T₃ vs. T₃ monomer in humans is shown in FIG. 10. In this study, twenty healthy male subjects were administered one of the drugs after a 10 hour overnight fast. The subjects were paired as closely as possible according to age, height and weight. The raw data from 10 subjects in each group tested for serum levels of total T₃ at 17 time points was used. Mean values were calculated for the 10 values at each time point as was the standard deviations. In order to compare the variability of the two groups at the same time points the standard deviations were divided by the mean values. The bars represent the values obtained so that a taller bar represents a greater variability.

It can be seen from this data that, for the time points where absorption is maximum (0.5-4 hours), the intersubject variability is greater for the T₃ monomer than it is for PolyT₃. The difference is greatest at 1, 1.5 and 2 hours after dosing which is the time period during which most absorption is taking place. However, it should be noted that the PolyT3 was administered as a solution while the T3 was administered as a tablet.

### Example 5

### Miscellaneous Examples of Conjugates

The following dipeptide conjugates of Furosemide were synthesized using the methods of the invention and include Boc-Ala-Ser(Furo)-Ome; Boc-Gly-Ser(Furo)-Ome; Boc-Leu-Ser(Furo)-Ome; Boc-Val-Ser(Furo)-Ome; Boc-Trp-Ser(Furo)-Ome; Boc-Cys-Ser(Furo)-Ome; Boc-Ile-Ser(Furo)-Ome; Boc-Met-Ser(Furo)-Ome; Boc-Phe-Ser(Furo)-Ome; Boc-Pro-Ser(Furo)-Ome; Boc-Arg-Ser(Furo)-Ome; Boc-Asp-Ser(Furo)-Ome; Boc-Glu-Ser(Furo)-Ome; Boc-His-Ser(Furo)-Ome; Boc-Lys-Ser(Furo)-Ome; Boc-Asn-Ser(Furo)-Ome; Boc-Gln-Ser(Furo)-Ome; Boc-Ser-Ser(Furo)-Ome; Boc-Thr-Ser(Furo)-Ome; Boc-Tyr-Ser(Furo)-Ome.

1. A method for altering bioavailability of a patient population to produce a serum profile described in FIG. 1 as compare to reference drug.

2. A method of reducing patient to patient variability through administering an orally active peptide-active agent composition.

3. The method of claim 1, wherein the composition improves AUC.

4. The method of claim 1, wherein the composition improves an active agent's facilitated diffusion rate as compared to the reference drug delivered alone.

5. The method of claim 1, wherein the composition improves an active agent's active transport as compared to the reference drug delivered alone.

6. The method of claim 1, wherein the composition improves an active agent's absorption as compared to the reference drug delivered alone.

7. The method of claim 1, wherein the composition improves an active agent's peak values as compared to the reference drug delivered alone.

8. A composition which provides the serum profile in FIG. 1.

9. A method of formulating a drug to reduce inter-subject variability comprising:
(i) a pharmaceutically effective agent; and
(ii) a peptide covalently bonded to said pharmaceutically active agent wherein said pharmaceutically effective agent is released according to a serum profile substantially identical to that of FIG. 1.

10. A method for controlling release of a pharmaceutically active agent to reduce inter-subject variability among a group of patients, comprising administering to said group of patients the composition according to claim 1.

11. A composition comprising:
(i) a pharmaceutically effective agent; and
(ii) a peptide covalently bonded to said pharmaceutically active agent wherein said pharmaceutically effective agent is released according to a serum profile substantially identical to that of FIG. 1.
